(19) United States
(12) Patent Application Publication
(10) Pub. No.: US 2002/0042599 A1
(43) Pub. Date: Apr. 11, 2002

(54) Flushable and anaerobically degradable films and laminates

(75) Inventors: Jean Jianqun Zhao, Cincinnati, OH (US); Saeed Fereshtehkhou, Cincinnati, OH (US); Jay William Gooch, Cincinnati, OH (US)

Correspondence Address: THE PROCTER & GAMBLE COMPANY, PATENT DIVISION, SHARON WOODS TECHNICAL CENTER- BOX C18, 11450 GROOMS ROAD, CINCINNATI, OH, 45242, US

(73) Assignee: The Procter & Gamble Company

(21) Appl. No.: 09/924,561

(22) Filed: Aug. 8, 2001

Related U.S. Application Data
(60) Provisional application No. 60/226,072, filed on Aug. 17, 2000.

(51) Int. Cl. A61F 13/20; A61F 13/15

(52) U.S. Cl. 604/367; 604/11; 604/370; 604/372

(57) ABSTRACT

The present invention relates to thermoplastic materials which are anaerobically degradable in an active sewage sludge. These materials are melt processable into fibers, films or laminates, and are suitable for use in an absorbent article, particularly flushable interlabial products, tampons and pantiliners.

### TECHNICAL FIELD

The present invention relates to flushable and anaerobically degradable films and laminates that are useful in disposable absorbent articles, particularly in tampons, interlabial devices, and pantiliners. These films and laminates are especially useful as the barrel and/or the plunger of a tampon applicator assembly, as the topsheet, the backsheet and/or the outer cover of other feminine hygiene products, and the wrappings for tampons or other feminine hygiene products.

### BACKGROUND OF INVENTION

Disposable absorbent articles such as feminine hygiene products, diapers, training pants, adult incontinence products, offer great convenience and are widely used by consumers. However, the popularity of these products has created a great concern regarding their disposal. Typical disposal methods such as incineration or landfill are costly and problematic to the environment. Therefore, there is a need for absorbent products that can be easily and cheaply disposed of without creating additional problems. An alternative disposal method has been proposed, which involves flushing the article down the conventional toilet and plumbing, subsequently, degrading it in the sewage system or septic system. For this disposal method, the suitable materials should not only have sufficient extensibility for the absorbent article application but also maintain the property and structural integrity during use. The suitable materials are easily flushed down the conventional toilet and pass through the plumbing system without creating blockage. More importantly, the suitable materials should degrade anaerobically in the sewage or septic system such that there is no accumulation of large chunks of the materials in the system. The last requirement is known to be the most challenging.

Water-soluble materials, such as polyethylene oxide, polyvinyl alcohol, are known to quickly lose the integrity when exposed to a large quantity of water, thus, they do not block the toilet and plumbing system, nor accumulate in the sewage or septic system. But these materials tend to be overly sensitive to the humid condition encountered during use and lose the strength prematurely (i.e., before disposal). Less water-soluble materials, used alone or in blends with the water-soluble materials, are capable of maintaining integrity and mechanical strength in use, but typically require additional treatments (such as acids, bases, enzymes) in the disposal system in order to disintegrate. Other materials, such as aliphatic polyesters or copolyesters, are known to be biodegradable under an aerobic condition. Yet, when these materials are exposed to the anaerobic condition typical of the septic/sewage system, they generally fail to degrade significantly within a reasonable amount of time such that no accumulation occurs in the system. Furthermore, it has been found that many biodegradable materials are prone to mold growth in hot and humid conditions, such as during shipping, storage and in-use.

Therefore, it is desirable to provide a flushable material that is degradable in the natural anaerobic sludge environment without further treatment in the disposal system. It is also desirable to provide a flushable and anaerobically degradable material that is melt extrudable or moldable such that it can be made into fibers, films, laminates or shaped articles, suitable for use in disposable absorbent articles, especially flushable products such as interlabial products, pantiliners or tampons. It is further desirable that such a material is soft and flexible to provide comfort to the wearer and minimize noises during wear. Additionally, it is desirable that such material has sufficient mechanical properties including the ability to be stretched and/or elongated without structural failures (e.g., tearing, ripping). It is also desirable that such material maintain its mechanical properties and remain mold-free until disposal.

### SUMMARY OF THE INVENTION

The present invention relates to materials which are anaerobically degradable in an active sewage sludge. These materials are melt processable into fibers, films, laminates or shaped articles, and are suitable for use in an absorbent article, particularly flushable interlabial products, tampons and pantiliners.

The materials comprise an anaerobically-responsive polymer and at least about 0.1 wt % of an inorganinc salts dispersed therein. The materials degrade anaerobically in an active sewage sludge over a 28 days period. In one embodiment, the degradation of the anaerobically degradable polymer of the present invention is characterized by (a) an increase in basis weight of at least about 10% and (b) a decrease in tensile elongation of at least about 30%, after being immersed in an active sludge medium for 28 days. In another embodiment, the degradation of blend of an aerobically degradable polymer and a water-responsive polymer is characterized by one or more of the following: (a) an increase in basis weight of at least about 5%; (b) a decrease in tensile elongation of at least about 20%; (c) a decrease in tensile elongation of at least about 30%, after being immersed in an active sludge medium for one hour.

These materials may be used alone or in blends with other polymers, such as water-responsive polymers. The materials or the blends thereof may be used as fibers, films, laminates, nonwoven webs, or shaped articles, and may be incorporated into at least a portion of the absorbent articles, such as topsheets, backsheets, outer covers, secondary layers, applicator assemblies, and wrappings.

### DETAILED DESCRIPTION OF THE INVENTION

Definitions

As used herein, the term “anaerobically degradable” means polymers, compositions, or articles made thereof (such as films, fibers, nonwovens, laminates, shaped articles) are capable of being degraded, weakened, broken into pieces, or dissolved, when immersed in an active sewage sludge obtained from a municipal waste water treatment plant/digester. Thus, the anaerobic degradation is manifested in one or more of the following: a change in the structure/composition of the material including changes in basis weight, molecular weight, inorganic content, or a loss of properties including mechanical properties such as tensile strength or elongation in the machine direction (MD) and/or the cross-machine direction (CD), water vapor impermeability, fluid impermeability, or a significant loss in structural integrity such as striation, fibrillation, cavitation, fragmentation.

As used herein, the term “water-responsive” means polymers, compositions, or articles made thereof (such as films, fibers, nonwovens, laminates, shaped articles) are capable of being weakened, broken into pieces, or dissolved when immersed in an aqueous medium and/or an aerobic environment, including water-soluble polymers and water-insoluble but aerobically degradable polymers.

As used herein, the term “flushable” means a material, film, laminate or product is capable of being flushed down a standard toilet without causing a blockage in the toilet and/or plumbing systems.

As used herein, the term “thermoplastic” means any polymeric material which can be softened under heat and/or pressure, and returned to its original state when heat and/or pressure is removed with little or no change in physical properties (assuming minimal oxidative degradation).

As used herein, the term “comprising” means the various components such as the films, layers, polymers, and materials, used in the present invention can be employed in various combinations and with other optional components, and that the various step used in the present invention can be used in various orders or combinations, so long as the objectives of the present invention are achieved. Accordingly, the term “comprising” encompasses the more restrictive terms “consisting essentially of” and “consisting of.”

As used herein, the term “water-soluble” means polymers or articles made thereof (such as fibers, films, nonwovens, laminates, shaped articles) are completely or substantially solubilized, dissolved or dispersed when exposed to an aqueous environment.

As used herein, “biodegradable” means polymers, films or articles that are capable of being degraded completely or substantially completely into carbon dioxide, water, biomass and inorganic materials by or in the presence of microorganisms.

All amounts, parts, ratios and percentages used herein are by weight unless otherwise specified.

Anaerobically Degradable Thermoplastic Polymers and Blends

Anaerobically degradable thermoplastic polymers useful in the present invention should exhibit one or more of the following characteristic degradations when immersed in an active sewage sludge: a change in the structure/composition of the material including changes in basis weight, molecular weight, inorganic content, or a loss of properties including mechanical properties such as tensile strength or elongation in MD and/or CD, water vapor impermeability, fluid impermeability, or a significant loss in structural integrity such as striation, fibrillation, cavitation, fragmentation. The anaerobically degradable polymers should preferably be melt processable by conventional plastic processes into fibers, films, nonwoven webs, laminates, or shaped articles. The anaerobically degradable polymers should have suitable mechanical properties and structural integrity desired for use in an absorbent article, such as extensibility, softness, flexibility and minimal noises. The anaerobically degradable polymers should also preferably be resistant to mold growth in a hot and humid environment, such as that typically present during storage, shipping, and wearing.

A variety of anaerobically degradable thermoplastic polymers are useful in the present invention including, but are not limited to, polyesteramides, polyhydroxyalkoates, and mixtures thereof. Aliphatic and partially aromatic polyesteramides are particularly preferred. These anaerobically degradable polymers are found to be degradable in an aerobic environment as well.

Aliphatic polyesteramides are prepared from various combinations of diols such as ethylene glycol, 1,4-butanediol, 1,3-propanediol, 1,6-hexanediol, and diethylene glycol; dicarboxylic acids such as oxalic acid, succinic acid, and adipic acid (or their respective esters); hydroxycarboxylic acids and lactones such as caprolactone; aminoalcohols such as ethanolamine and propanolamine, cyclic lactams such as ε-caprolactam or lauric lactam; omega-aminocarboxylic acids such as aminocaproic acid; mixtures (1:1 salts) of dicarboxylic acids such as adipic acid and succinic acid and diamines such as hexamethylenediamine and diaminobutane; and hydroxy-terminated or acid-terminated polyesters with molecular weights from about 200 to about 10,000; as well as compatible mixtures or blends of these polymers, such as, for example, poly(tetramethylene succinate-co-terephthalate) copolyesters, poly(tetramethylene glutarate-co-terephthalate) copolyesters, poly(tetramethylene terephthalate-co-diglycolate) copolyesters, poly(tetramethylene glutarate-co-naphthalate) copolyesters and poly(tetramethylene) adipate-co-terephthalate copolyesters. Detailed description of the aliphatic polyesteramides can be found in U.S. Pat. No. 5,644,020, issued to Timmerman et al. on Jul. 1, 1997, the disclosure of which is hereby incorporated by reference.

Suitable polyesteramides typically comprise from about 20 to about 80 wt % ester and from about 20 to about 80 wt % amide, preferably from about 30 to about 50 wt % ester and from about 50 to about 70 wt % amide. Suitable polyesteramides typically have an averaged molecular weights (Mw) of from about 50,000 to about 200,000. Suitable polyesteramides are preferably semi-crystalline with a melting temperature in the range of from about 90 to about 190° C., preferably from about 100 to about 185° C., and more preferably from about 110 to about 180° C.

Aliphatic polyesteramides that are particularly preferred for use in the present invention include, but are not limited to, those prepared from combinations of adipic acid, butanediol or hexanediol, and aminocaproic acid or ε-caprolactam. The preferred polyesteramides are available from Bayer under the BAK 402, 403 and 404 designations.

Also useful herein are polyhyroxyalkanoate polymers and copolymers including polyhydroxybutyrate polymers and polyhydroxybutyrate/valerate copolymers disclosed in U.S. Pat. No. 5,391,423, issued to Wnuk et al. on Feb. 21, 1995, and is hereby incorporated by reference. Other nonlimiting examples of polyhydroxyalkanoates include poly(3-hydroxybutyrate-co-3-hydroxyhexanoate), poly(3-hydroxybutyrate-co-3-hydroxyoctanoate), poly(3-hydroxybutyrate-co-3-hydroxynonanoate), poly(3-hydroxybutyrate-co-3-hydroxydecanoate), poly(3-hydroxybutyrate-co-3-hydroxydocosanoate), poly(3-hydroxybutyrate-co-3-hydroxyhexadecanoate), poly(3-hydroxyvalerate-co-3-hydroxyoctanoate), poly(3-hydroxybutyrate-co-3-hydroxyvalerate-co-3-hydroxyoctanoate), poly(3-hydroxybutyrate-co-3-hydroxyvalerate-co-3-hydroxydecanoate), and poly(3-hydroxybutyrate-co-3-hydroxyvalerate-co-3-hydroxyoctanoate-co-3-hydroxydecanoate) copolymers disclosed in U.S. Pat. No. 5,489,470 issued Feb. 6, 1996, and U.S. Pat. No. 5,498,692, issued Mar. 12, 1996, both of which are issued to Noda and are hereby incorporated by reference.

Also useful in the present invention are blends of the anaerobically degradable thermoplastic polymers and water-responsive thermoplastic polymers. The inclusion the water-responsive thermoplastic polymers allows the blended material to dissolve and lose integrity more quickly after the material is disposed in the toilet and/or the sewage/septic system. A variety of water-responsive thermoplastic polymers can be used in blends. The suitable water-responsive polymers should provide the resultant blends with the desired properties such as melt processability, mechanical properties and structural integrity during wear, mold resistance, and anaerobic degradability.

Water-responsive polymers useful in the present invention may include water-soluble polymers. Nonlimiting examples of water-soluble polymers include hydroxyalkyl cellulose, particularly hydroxypropyl cellulose, alkyl hydroxypropyl cellulose, polyethylene oxide, polypropylene oxide, poly(ethylene-propylene) oxide, polyvinyl alcohol and polyvinyl alcohol copolymers, polyvinylpyrolidone, polyvinyl pyridine, gelatinized starch, and interpenetrated networks of starch with ethylene/vinyl alcohol copolymers disclosed in U.S. Pat. No. 5,391,423 (Wnuk et al), issued Feb. 21, 1995 (herein incorporated by reference), nylon copolymers, acrylic acid copolymers, polyethylene glycol, as well as compatible mixtures and blends of these polymers.

Particularly preferred water-soluble polymers include polyethylene oxide available from Union Carbide under the designation Polyox® WSRN-10 (Mw 100,000), WSRN-80 (Mw 200,000) and WSRN-750 (Mw 300,000), and polyvinyl alcohol available from Air Products under the designation Vinex® 1090, 2034, 2025, 2144 and 5030.

Water-responsive polymers useful in the present invention may also include a variety of biodegradable polymers which have limited to no solubility in water. The biodegradation potential can be estimated by measuring carbon dioxide evolution and dissolved organic carbon removal from a medium containing the substance being tested as the sole carbon and energy source and a dilute bacterial inoculum obtained from the supernatant of homogenized activated sludge. See Larson, “Estimation of Biodegradation Potential of Xenobiotic Organic Chemicals,” Applied and Environmental Microbiology, Volume 38 (1979), pages 1153-61, which describes a suitable method for estimating biodegradability. These polymers are primarily degradable in an aerobic environment. Although not required, anaerobically degradability of these polymers are also desirable.

Water-insoluble, biodegradable polymers useful in the present invention include poly(lactic acid) polymers; polycaprolactones disclosed in U.S. Pat. No. 5,391,423 (Wnuk et al), issued Feb. 21, 1995 (herein incorporated by reference); aliphatic polyesters; aliphatic polyalkylene succinate polymers, polyalkylene succinate adipate copolymers or mixtures thereof disclosed in U.S. Pat. No. 5,849,401 (El-Afandi et al), issued Dec. 15, 1998 and U.S. Pat. 5,910,545 (Tsai et al), issued Jun. 8, 1999 (herein incorporated by reference); aliphatic-aromatic copolyesters preferably comprising 10 to 1000 repeating units, most preferably from 15 to 600 repeating units, disclosed in U.S. Pat. No. 5,292,783 (Buchanan et al), issued Mar. 8, 1994, U.S. Pat. No.5,446,079 (Buchanan et al), issued Aug. 29, 1995, U.S. Pat. No. 5,559,858 (Buchanan et al), issued Feb. 4, 1997, and U.S. Pat. No. 5,580,911 (Buchanan et al), issued Dec. 3, 1996 (herein incorporated by reference) that are prepared from combinations of dicarboxylic acids or derivatives thereof including those selected from malonic, succinic, glutaric, adipic, pimelic, azelaic, sebacic, fumaric, 2,2-dimethyl glutaric, suberic, 1,3-cyclopentanedicarboxylic, 1,4-cyclohexanedicarboxylic, 1,3-cyclohexanedicarboxylic, diglycolic, itaconic, maleic, 2,5-norbornanedicarboxylic, 1,4-terephthalic, 1,3-terephthalic, 2,6-naphthoic, and 1,5-naphthoic acid, and ester forming derivatives thereof, and combinations thereof, and diols selected from ethylene glycol, diethylene glycol, propylene glycol, 1,3-propanediol, 2,2-dimethyl-1,3-propanediol, 1,3-butanediol, 1,4-butanediol, 1,5-pentanediol, 1,6-hexanediol, 2,2,4-trimethyl-1,6-hexanediol, thiodiethanol, 1,3-cyclohexanedimethanol, 1,4-cyclohexanedimethanol, 2,2,4,4-tetramethyl-1,3-cyclobutanediol, triethylene glycol, tetraethylene glycol, and combinations thereof.

Particularly preferred water-insoluble, biodegradable polymers for use in the present invention that are relatively easy to process into films by conventional techniques and have particularly desirable mechanical properties include poly(lactic acid) polymers; polycaprolactones; aliphatic polyalkylene succinate polymers, polyalkylene succinate adipate copolymers or mixtures thereof; and aliphatic-aromatic copolyesters. Particularly suitable for use in the present invention are polybutylene succinate polymers and polybutylene succinate adipate copolymers having weight-averaged molecular weights (Mw) of from about 40,000 to about 300,000 with a degree of polydispersity (Mw/Mn) in the range of from about 1.8 to about 3.6 (available from Showa Highpolymer Co. Ltd, Tokyo, Japan, under the Bionolle Type 1000 and 3000 designations). Also suitable for use in the present invention are poly(tetramethylene) adipate-co-terephthalate copolyesters (available from Eastman Chemical under the Eastar Biodegradable Copolyester 14776 designation).

The composition of the blends suitable for use herein will depend on the particular polymers involved, the properties, in particular the desired rate of degradation and disintegration in an anaerobic environment such as an active sewage sludge, the intended use of the film and like factors. The blend typically comprises from about 50 to 100 wt % of an anaerobically degradable thermoplastic polymer and from 0 to about 50 wt % of a water-responsive thermoplastic polymer. Preferably, the blend comprises from about 60 to about 95 wt % of an anaerobically degradable polymer, and from about 5 to about 40 wt % of a water-responsive polymer. More preferably, the blend comprises from about 70 to about 90 wt % of an anaerobically degradable polymer, and from about 10 to about 30 wt % of a water-responsive polymer.

Composition and Characteristics of the Anaerobically Degradable Material

The anaerobically degradable materials (polymers or blends) of the present invention may comprise at least about 0.1 wt % inorganic salts. Nonlimiting examples of the inorganic salts include metal carbonates, metal oxides, metal phosphates, metal chlorides, metal sulfates, and mixtures thereof. Representative metal cations in these inorganic salts may include calcium, potassium, sodium, magnesium, other I_{A} and II_{A} metal cations, aluminum, titanium and silicon. Particularly preferred inorganic salts include calcium carbonate, magnesium carbonate, potassium carbonate, sodium carbonate, calcium chloride, magnesium chloride, calcium phosphate, titanium oxide, silicone oxide, aluminum oxide, and mixtures thereof. Typically, the inorganic salt content ranges from about 0.1 to about 60 wt %, preferably from about 1 to about 50 wt %, and more preferably from about 2 to about 40 wt %. In one embodiment, the anaerobically degradable material comprises from about 1 to about 10 wt % calcium carbonate. In another embodiment, the anaerobically degradable material comprises from about 1 to about 20 wt % of a mixture of calcium carbonate and titanium oxide, wherein the CaCO3/TiO2 ratio ranges from 10:1 to 1:10, preferably from 5:1 to 1:5, and more preferably 1:1. Particularly preferred embodiments comprise aliphatic polyesteramides and calcium carbonates or calcium carbonate/titanium oxide mixtures Optionally, it may be desirable to incorporate one or more additives into the anaerobically degradable material of the present invention. Suitable additives include, but are not limited to, processing aids, fillers, surfactants, plasticizers, compatibilizers, impact modifiers, nucleating agents, anti-oxidants, heat or ultraviolet stabilizers, colorants, anti-static agents, lubricants, blowing agents, dispersants, thickening agents, antimicrobials, and mixtures thereof. Typically these additives comprise up to about 10 wt %, preferably up to about 20 wt %, and more preferably up to about 30 wt %, of the anaerobically degradable composition of the present invention.

In one embodiment, a wax is incorporated into the anaerobically degradable material to modify the viscosity, and to improve processability. Nonlimiting examples of wax include amide waxes, ester waxes, natural waxes, synthetic waxes, paraffin waxes, isoparaffin waxes, microcrystalline waxes, and mixtures thereof. In a preferred embodiment, a polar wax, such as ester wax or amide wax, is incorporated in an amount less than about 5 wt %, preferably less than about 2.5 wt %, and more preferably less than about 1 wt %. In another preferred embodiment, a polar wax, such as ester wax or amide wax, is incorporated in an amount ranging from about 0.1 to about 1 wt %.

After immersion in an active sewage sludge for days, the films made of the anaerobically degradable thermoplastic polymers of the present invention exhibit a loss in structural integrity. Under a microscope, one observes openings in the recovered sample films. Most of these openings have an elongated shape with the long axis oriented substantially in the machine direction (MD). At the end of the 28 days immersion, some film samples exhibit severe loss in structural integrity such that sections of the resultant samples are broken into ribbons and fibrils, or cavitated substantially through the thickness of the sample.

Surprisingly, these observed structural breakdowns are accompanied by a weight gain (i.e., an increase in basis weight). An anaerobically degradable polymer useful in the present invention should typically has a weight gain (as measured according to the Test Method below) of at least about 10%, preferably at least about 15%, and more preferably at least about 25%, after 28 days immersion in an active sewage sludge. Typically, the weight gain is measurable by 14 days immersion in the sludge. At the end of the 28 days test, a weight gain of 35 wt % may be obtained. The weight gain is often accompanied by an increase in inorganic content, particularly calcium.

The anaerobically degradable polymeric material suitable for use herein may show decreases in tensile properties, such as tensile strength and tensile elongation at break in the machine direction (MD) and/or in the cross machine direction (CD). Suitable materials typically have a decrease in tensile strength of at least about 30%, preferably at least about 40%, more preferably at least about 50%, and/or a decrease in tensile elongation of at least about 30%, preferably at least about 50%, and more preferably at least about 75%, and most preferably at least about 85%, after 28 days immersion in an active sewage sludge and measured according to the Test Method below.

For semi-crystalline polymers, the orientation within the sample film, which may be process-dependent, affects the changes in tensile properties. In one embodiment, the recovered cast film, having more orientation along the MD, shows a greater decrease in CD tensile properties than in MD tensile properties. In another embodiment, the recovered blown film does not show as much difference in MD versus CD tensile property degradation.

Molecular weight of the polymer also affects the anaerobic degradability. Lower molecular weight polymers degrades more readily and more completely than the higher molecular weight ones of the same polymers. In one embodiment, suitable aliphatic polyesteramide has a relative viscosity in the range of from about 2.2 to about 3.5, preferably from about 2.6 to about 2.9.

Polymeric blends suitable for use herein may have degradation properties different from those of anaerobically degradable polymers alone, depending on the composition, the characteristics of the other components in the blend, etc. Typically, the suitable blend materials should have one or more of the following degradation characteristics: a weight change of at least about 5%, preferably at least about 10% and more preferably at least about 20%; or a decrease in tensile strength of at least about 20%, preferably at least about 30% and more preferably at least about 40%; or a decrease in tensile elongation of at least about 30%, preferably at least about 40% and more preferably at least about 50%, after immersion in an active sewage sludge, after immersion in an active sewage sludge, and recovered at sampling time of one hour, preferably 4 hours, more preferably 8 hours and most preferably 24 hours, and measured according to the Test Method below.

Composition and Characteristics of the Laminate

In a preferred embodiment, a laminate of the present invention comprises: (1) a substantially anaerobically degradable layer comprising the anaerobically degradable material disclosed herein; (2) a substantially water-soluble layer adjacent the anaerobically degradable layer; and (3) optionally, a substantially water-permeable layer adjacent the water-soluble layer. Each layer of the laminate is a film or a web, either co-extruded to form a laminate, or made separately prior to being combined into a laminate, or a combination thereof.

In one embodiment, the water-soluble layer is substantially thicker than the other two layers. As used herein, “substantially thicker” means that the water-soluble layer is sufficiently thicker relative to the thickness of each of the anaerobically degradable and water-permeable layers such that the laminate, after it is flushed, will eventually and preferably rapidly lose integrity as the relatively thick water-soluble layer is dissolved and disintegrated, leaving behind the relatively thin anaerobically degradable and water-permeable layers that take up a significantly smaller volume. Typically, the water-soluble layer is at least about 2 times as thick as each of the anaerobically degradable and water-permeable layers. Preferably, the water-soluble layer is at least about 3 times as thick as the anaerobically degradable and water-permeable layers.

For laminates of the present invention which do not comprise the optional water-permeable layer, the water-soluble layer typically comprises from about 70 to about 95%, preferably from about 80 to about 90%, of the thickness of the entire laminate, while the anaerobically degradable layer comprises from about 5 to about 30%, preferably from about 10 to about 20%, of the thickness of the entire laminate. For laminates of the present invention which do comprise the optional but preferred water-permeable layer, the water-soluble layer typically comprises from about 50 to about 95%, preferably from about 60 to about 80%, of the thickness of the entire laminate, while the anaerobically degradable and water permeable layers each comprise from about 2.5 to about 25%, preferably from about 5 to about 20%, of the thickness of the entire laminate.

The laminate of the present invention can be prepared to any desired thickness, so long as they remain water-flushable and biodegradable. In the case of backsheets for disposable absorbent articles, such laminates are relatively thin. Suitable laminates for such backsheets typically have a thickness of from about 0.5 to about 3 mil (13-76μ). Preferably, such laminates have a thickness of from about 0.7 to about 1.6 mil (18-41μ).

Within the constraints defined hereinabove, the thickness of each of the layer may vary. Typically the anaerobically degradable layer has a thickness of from about 0.05 to about 0.5 mil, preferably from about 0.1 to about 0.3 mil. For laminates of the present invention which do not comprise the optional water-permeable layer, the water-soluble layer typically has a thickness of from about 0.3 to about 1.5 mil (8-38μ), preferably from about 0.6 to about 1.3 mil (15-33μ). For laminates of the present invention which do comprise the optional water-permeable layer, the water-soluble layer typically has a thickness of from about 0.5 to about 1.2 mil (13-30μ), preferably from about 0.6 to about 1.0 mil (15-25μ). Where incorporated, the water-permeable layer has a thickness of from about 0.05 to about 0.5 mil (1-13μ). Preferably, this water-permeable layer has a thickness of from about 0.1 to about 0.3 mil (2-8μ).

The water-soluble layer comprises from about 60 to 100 wt % of a substantially water-soluble thermoplastic polymer as previously defined, and from 0 to about 40 wt % of a substantially water-insoluble biodegradable thermoplastic polymer as previously defined. The particular amounts used will depend on the particular polymers involved, the properties, in particular the rate of degradation and disintegration desired in the presence of an aqueous environment such as water, the intended use of the laminate and like factors. The inclusion of a minor amount of water-insoluble thermoplastic biodegradable polymer allows this water-soluble layer to have improved mechanical properties and to maintain sufficient integrity during use before flushing, yet allows this layer to dissolve and lose integrity after the laminate is flushed. Typically, the water-soluble layer comprises from about 60 to about 95 wt % of a water-soluble polymer, and from about 5 to about 40 wt % of a water-insoluble biodegradable polymer. Preferably, the water-soluble layer comprises from about 70 to about 90 wt % of a water-soluble polymer, and from about 10 to about 30 wt % of a water-insoluble biodegradable polymer.

The water-permeable layer comprises from about 30 to about 70 wt % of a substantially water-soluble thermoplastic polymer as previously defined, and from about 30 to about 70 wt % of a substantially water-insoluble biodegradable thermoplastic polymer as previously defined. The particular amounts used will depend on the particular polymers involved, the properties, in particular the aqueous liquid control properties desired, the intended use of the laminate and like factors. In order for this layer to control the rate at which aqueous liquids pass through to the adjacent water-soluble layer, the amount of water-soluble thermoplastic polymer in the water-soluble layer needs to be greater than the amount of water-soluble thermoplastic polymer in the water-permeable layer. Preferably, the water-permeable layer comprises from about 40 to about 60 wt % of a water-soluble polymer, and from about 40 to about 60 wt % of a water-insoluble biodegradable polymer.

The laminates of the present invention are especially suitable for use in disposable absorbent articles. As used herein, the term “absorbent articles” refers to articles that absorb and contain aqueous body liquids, and more specifically refers to articles that are placed against or in proximity to the body of the wearer to absorb and contain the various aqueous liquids discharged from the body. Additionally, the term “disposable absorbent articles” refers to articles which are intended to be discarded after a single use (i.e., the original absorbent article in its whole is not intended to be laundered or otherwise restored or reused as an absorbent article, although certain materials or all of the absorbent article can be recycled, reused, composted or flushed). The present invention is applicable to various absorbent articles such as diapers, incontinent briefs, incontinent pads, training pants, pull-on diapers, diaper inserts, catamenial pads, sanitary napkins, pantiliners, interlabial devices, tampons, facial tissues, paper towels, breast pads, and the like, as well as other potentially flushable items, such as tampon applicator assemblies (including the barrel and the plunger), tampon cords, wrappers and packaging for various products, including disposable absorbent articles, disposable gloves and the like.

These absorbent articles typically comprise a substantially water-impervious backsheet made from the film of the present invention, a substantially water-permeable topsheet joined to, or otherwise associated with the backsheet, and an absorbent core positioned between the backsheet and the topsheet. The topsheet is positioned adjacent to the body-facing surface of the absorbent core. The topsheet is preferably joined to the absorbent core and to the backsheet by attachment means such as those well known in the art. As used herein, the term “joined” encompasses configurations whereby an element is directly secured to the other element by affixing the element directly to the other element, and configurations whereby the element is indirectly secured to the other element by affixing the element to intermediate member(s) which in turn are affixed to the other element. In preferred absorbent articles, the topsheet and the backsheet are joined directly to each other at the periphery thereof. The topsheet and backsheet can also be indirectly joined together by directly joining them to the absorbent core by the attachment means.

Detailed description of the laminate and the disposable absorbent articles can be found in U.S. patent application Ser. No. 09/520,676, filed Mar. 7, 2000 by Zhao et al., the disclosure of which is hereby incorporated by reference.

Test Methods

1. Weight Changes During Anaerobic Degradation

This test determines the extent to which a product disintegrates upon exposure to biologically active anaerobic sludge. Anaerobic conditions are typically found in household septic tanks, as well as in municipal sewage treatment facilities in the form of anaerobic sludge digesters.

The anaerobic sludge used in this test is obtained from a municipal waste water treatment plant. The sludge is poured through a 1 mm sieve to removed any large solids. The sludge should meet the following criteria for use in the test:

pH between 6.5 and 8;

total solids ≧15,000 mg/L;

total volatile solids ≧10,000 mg/L;

wherein the term “total volatile solids” means the solid matters in the sludge that are organic, as opposed to inorganic, in nature.

The test procedure is as follows:

1. the test samples and the controls (100% cotton TAMPAX® tampons) are preconditioned in a hot air oven at 103°±2° C. for 2 hours.

2. each sample/control is weighed, then placed in a 2 L reactor bottle filled with 1200 ml anaerobic sludge; a triplicate set for each sample/control per sampling time point is prepared;

3. the reactor bottle is capped with a latex stopper having one-hole therein to allow for venting of evolved gases, and placed in a 35° C. incubator;

4. on the designated sampling time, the contents of each reactor will be passed through a 1 mm screen to recover any undisintegrated material;

5. any collected material will be rinsed with tap water to remove the sludge;

6. visual observations of the physical appearance of the materials when recovered from the reactors will be made and recorded;

7. sample residues are collected off the screen and placed in a disposable beaker for drying in a hot air oven at 103±2° C. for at least 2 hours, or at a lower temperature overnight; and

8. the dried material will be weighed to determine final weight.

The rate and extent of anaerobic disintegration of each test material and the control material is determined from initial dry weights of the material and the dried weights of the material recovered on the sampling days. The percent anaerobic disintegration is determined using the following equation (percent weight gain):

$$Percent Disintegration = \frac{( final dry weight - initial dry weight )}{( initial dry weight )} \times 100$$

The average percent disintegration for the test sample and control for each sampling time point will be reported.

The criteria for the activity of the sludge requires that the control tampon material must lose at least 95% of its initial dry weight after 28 days exposure.

2. Tensile Strength and Elongation at Break

A commercial tensile tester from Instron Engineering Corp., Canton, Mass. or SINTECH-MTS Systems Corporation, Eden Prairie, Minn. may be used for this test. For CD tensile properties, the films or laminates are cut into 1″ wide in MD (the machine direction of the film/laminate) by 4″ long in CD (the cross machine direction which is at a 90° angle from MD) specimens. For MD tensile properties, the orientation of the film/laminate is rotated 90°. The instrument is interfaced with a computer for controlling the test speed and other test parameters, and for collecting, calculating and reporting the data. The tensile properties of the samples are determined according to ASTM Method D882-95a.

These tensile properties are measured at room temperature (about 20° C.). The samples tested include the original sample and the samples recovered at certain sampling time points. The procedure is as follows:

1. choose appropriate jaws and load cell for the test; the jaws should be wide enough to fit the sample, typically 1″ wide jaws are used; the load cells is chosen so that the tensile response from the sample tested will be between 25% and 75% of the capacity of the load cells or the load range used, typically a 50 lb load cell is used;

2. calibrate the instrument according to the manufacture's instructions;

3. set the gauge length at 2″;

4. place the sample in the flat surface of the jaws according to the manufacture's instructions;

5. set the cross head speed at a constant speed of 20″/min;

6. start the test and collect data simultaneously; and

7. calculate and report tensile properties including elongation at break and peak load. The average result of a triplicate set of each sample is reported.

3. Resistance to Mold Based on Water Activity

The resistance to mold for the films of the present invention is based on visual signs of mold growth on the film during storage in an extreme hot/humid environment. Approximately 0.2 ml of a medium containing mold spores (1.0×10⁴ cfu/ml) is dispensed directly onto the film. The film is then placed in a 26.7° C. environment having a relative humidity of 80% for 2 weeks. Film samples having no visible signs of mold growth after 2 weeks are considered to be resistant to mold growth.

### EXAMPLES

### Example 1

A polymer composition of polyesteramide BAK 404, the high molecular weight fraction (having a relative viscosity of about 3.2, according to the manufacturer), 0.1 wt % amide wax, 6 wt % CaCO3 and 6 wt % TiO2 is made into a cast film (about 0.9 mil in thickness) using a conventional thermoplastic extruder.

### Example 2

A polymer composition of polyesteramide BAK 404, the low molecular weight fraction (having a relative viscosity of about 2.8, according to the manufacturer), 0.1 wt % amide wax, 6 wt % CaCO3 and 6 wt % TiO2 is made into a cast film (about 0.7 mil in thickness) using a conventional thermoplastic extruder.

### Example 3

A polymer composition of polyesteramide BAK 403, 6 wt % CaCO3 and 6 wt % TiO2 is made into a cast film (about 0.9 mil in thickness) using a conventional thermoplastic extruder.

### Comparative Examples 4a and 4b

Biodegradable, water-insoluble polymer compositions of an aliphatic polyester Bionelle 3001 (example 4a) and an aliphatic-aromatic copolyester Eastar 14766 (example 4b) are made into cast films (about 0.7-0.9 mil in thickness) using a conventional thermoplastic extruder.

### Example 5

Films of Examples 1-3 are tested for tensile properties according to the Test Method described herein. The properties of the films are shown in the following Table:

| EXAMPLE | 1 | 2 | 3 |
|---|---|---|---|
| MD Tensile at Peak Load (Gm) | 2110 | 2450 | 3600 |
| MD Elongation (%) | 120 | 80 | 90 |
| CD Tensile at Peak Load (Gm) | 1480 | 800 | 1860 |
| CD Elongation (%) | 550 | 440 | 590 |

The results show that the examples of the present invention have satisfactory tensile properties suitable for use in an absorbent article.

### Example 6

Films of examples 1-3 and comparative examples 4a and 4b are immersed in an active sewage sludge for up to 28 days, and the recovered samples are tested for weight change, tensile properties, inoculated mold growth, according to the Test Method described herein.

The following Table shows the changes in CD elongation at break and (standard Deviation SD) when the samples immersed in the sludge are tested at sampling time points: 0 day, 7 days, 14 days and 28 days:

| EXAMPLE | Day 0 (SD) | Day 7 (SD) | Day 14 (SD) | Day 28 (SD) |
|---|---|---|---|---|
| 1 | 550 | 40 | 20 | 30 |
| 2 | 440 (52) | 20 | 20 (4) | 10 (4) |
| 3 | 590 (58) | 40 (15) | 10 (50) | 60 (39) |
| 4a | 710 (67) | — | — | 140 (20) |
| 4b | 900 (50) | — | — | 910 (90) |

The results show that Comparative Examples which are water-insoluble and aerobically degradable do not provide satisfactory degradability in an anaerobic sludge. The results also show that Examples 1-3 of the present invention degrades significantly in an anaerobic sludge after just 7 days.

The following Table shows the properties of Examples 1-3 after 28 days immersion in an active sewage sludge:

| EXAMPLE | 1 | 2 | 3 |
|---|---|---|---|
| 28 Days Weight Gain (%) | 30 | 13 | 11 |
| Inoculated Molding | no | no | no |
| Water Activity* | — | 0.60 | 0.59 |

*a material having a water activity <0.7 would not support mold growth thereon.

The results show that Examples 1-3 of the present invention gain weight in the anaerobic degradation process. The results also show that Examples 1-3 of the present invention have water activity values lower than 0.7, thus, there is no inoculated molding on these sample films.

### Example 7

A polymer composition of polyesteramide BAK 404, the high molecular weight fraction (having a relative viscosity of about 3.2, according to the manufacturer), 0.1 wt % amide wax, 6 wt % CaCO3 and 6 wt % TiO2 is made into a blown film (about 0.9 mil in thickness) using a conventional thermoplastic film blowing equipment.

Films of samples 1 and 7 are immersed in an active sewage sludge for up to 28 days; the samples are recovered at sampling time points 0 day, 7 days and 28 days, and the recovered samples are tested for inorganic content, particularly calcium, using AA (Atomic Absorption Spectroscopy); residues using TGA (Thermal Gravimetric Analysis); and weight gain. The results are shown in the following Table:

| | Sampling | Ca by | *CaCO3 | Weight | |
|---|---|---|---|---|---|
| | Time | AAS | Equivalent | Gain | TGA Residue |
| EXAMPLE | (days) | (wt %) | (wt %) | (wt %) | (wt %) |
| 1 | 0 | 2.17 | 5.37 | — | 15.42 |
| | 7 | 4.59 | 11.7 | — | 17.39 |
| | 14 | 7.35 | 18.35 | — | 21.08 |
| | 28 | 13.75 | 34.31 | 30 | 35.06 |
| 7 | 0 | 1.99 | 4.99 | — | 11.89 |
| | 7 | 5.77 | 14.43 | — | 23.67 |
| | 28 | 12.44 | 31.10 | 34 | 36.36 |

*The CaCO3 equivalent is calculated from the Ca content detected by AAS and the assumption that all Ca are in the carbonate form.

The results show an increase of calcium deposits on the degrading film samples with time, and a good correlation with the weight gain. The results of the TGA residues show that the recovered film samples may contain other inorganic matters or non-carbonaceous substances.

The disclosures of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A laminate comprising at least one anaerobically degradable layer, wherein the anaerobically degradable layer has:
(a) an increase in basis weight of at least about 10%; and
(b) a decrease in tensile elongation of at least about 30% after being immersed in an active anaerobic sludge medium for 28 days.

2. The laminate of claim 1 wherein the anaerobically degradable layer comprises an anaerobically degradable polymer and at least about 0.1 wt % of inorganinc salts.

3. The laminate of claim 2 wherein the anaerobically-degradable polymer is selected from the group consisting of polyestersamides, polyhydroxyalkoates, and mixtures thereof.

4. The laminate of claim 3 wherein the polyesteramide has a melting temperature in the range of from about 90° C. to about 190° C.

5. The laminate of claim 3 wherein the polyesteramide comprises from about 20 to about 80 wt % ester and from about 20 to about 80 wt % amide.

6. The laminate of claim 5 wherein the polyesteramide comprises from about 30 to about 50 wt % ester and from about 50 to about 70 wt % amide.

7. The laminate of claim 2 wherein the anaerobically degradable layer comprises from about 0.1 wt % to about 60 wt % of inorganic salts comprising metal ions selected from the group consisting of calcium, magnesium, sodium, potassium, titanium, silicon, aluminum, and mixtures thereof.

8. The laminate of claim 7 wherein the inorganinc salts are selected from the group consisting of calcium carbonate, magnesium carbonate, potassium carbonate, sodium carbonate, calcium chloride, magnesium chloride, calcium phosphate, titanium oxide, silicone oxide, aluminum oxide, and mixtures thereof.

9. The laminate of claim 2 wherein the anaerobically degradable layer further comprises processing aids, fillers, surfactants, plasticizers, compatibilizers, impact modifiers, nucleating agents, anti-oxidants, heat or ultraviolet stabilizers, colorants, anti-static agents, lubricants, blowing agents, dispersants, thickening agents, antimicrobials, and mixtures thereof.

10. The laminate of claim 1 wherein the anaerobically degradable layer is resistant to mold growth.

11. A laminate comprising at least one anaerobically degradable layer, wherein the anaerobically degradable layer comprises an anaerobically degradable polymer and a water-responsive polymer and the anaerobically degradable layer has one or more of the following:
(a) a change in basis weight of at least about 5%;
(b) a decrease in tensile strength of at least about 20%;
(c) a decrease in tensile elongation of at least about 30% after being immersed in an active anaerobic sludge medium for one hour.

12. The laminate of claim 11 wherein the anaerobically degradable layer comprises from about 50 to about 100 wt % of the anaerobically degradable polymer and from about 0 to about 50 wt % of the water-responsive polymer.

13. The laminate of claim 12 wherein the anaerobically degradable layer comprises from about 60 to about 95 wt % of the anaerobically degradable polymer and from about 5 to about 40 wt % of the water-responsive polymer.

14. The laminate of claim 11 wherein the anaerobically degradable polymer is selected from the group consisting of polyesteramide, polyhydroxyalkoate, and mixtures thereof, and the water-insoluble biodegradable polymer is selected from the group consisting of polyvinyl alcohol, polyethylene oxide, polypropylene oxide, poly(ethylene-propylene) oxide, poly(lactic acid), polycaprolactone, aliphatic-aromatic copolyester, polyalkylene succinate, polyalkylene succinate adipate, starch and derivatives, hydroxyalkylcellulose, alkyl hydroxypropyl cellulose, and mixtures thereof.

15. The laminate of claim 11 wherein the anaerobically degradable layer further comprises at least about 0.1 wt % of inorganinc salts.

16. The laminate of claim 15 wherein the anaerobically degradable layer comprises from about 0.1 wt % to about 60 wt % of inorganic salts comprising metal ions selected from the group consisting of calcium, magnesium, sodium, potassium, titanium, silicon, aluminum, and mixtures thereof.

17. An absorbent article comprising a topsheet, a backsheet and an absorbent core disposed between the topsheet and the backsheet, wherein at least a portion of the topsheet or the backsheet comprises the laminate of claim 11.

18. A tampon applicator assembly comprising a barrel and a plunger, wherein at least a portion of the barrel or the plunger comprises the laminate of claim 11.
